(19) United States
(12) Patent Application Publication
(10) Pub. No.: US 2005/0171418 A1
(43) Pub. Date: Aug. 4, 2005

(54) Capsule endoscopy system

(76) Inventor: Tah-Yeong Lin, Tao yuan (TW)

Correspondence Address: J.C. Patent, Inc., Suite 250, 4 Venture, Irvine, CA, 92618, US

(21) Appl. No.: 10/753,719

(22) Filed: Jan. 8, 2004

(51) Int. Cl. A61B 5/05; A61B 1/04; A61B 1/06

(52) U.S. Cl. 600/407; 600/109; 600/160

(57) ABSTRACT

A capsule endoscopy system, comprising a capsule endoscopy, a data recorder and an image processor, is disclosed. The capsule endoscopy for catching images of the digestive tract and transforming into an image data comprises a first transceiver. The data recorder comprises a second transceiver, a third transceiver and a memory coupled to the second and the third transceivers. The image data are transmitted from the first transceiver to the second transceiver and the image data received by the second transceiver is stored in the memory. In addition, the image data stored in the memory is transmitted from the third transceiver to the image processor.

### BACKGROUND OF THE INVENTION

1. Field of the Invention

The present invention relates to a capsule endoscopy system, and more particularly to a capsule endoscopy system immediately transmitting the images of a digestive tract to a display.

2. Description of the Related Art

Vessel diseases, digestive diseases and cancers are the main concerns for human being. Traditionally, endoscopies have been widely used for observing the digestive system in medical treatments. However, the endoscopies cannot catch the images in the digestive system, such as small intestines. More worse, patients do not feel comfortable during the medical processes. In order to resolve the problems, capsule endoscopies have been widely used in the industry.

FIGS. 1 and 2 are transmission configuration of the prior art capsule endoscopy system. Referring to FIGS. 1 and 2, the prior art capsule endoscopy system 100 comprises: a capsule endoscopy 110, a data recorder 120, an image processor 130 and a display 140. Generally, the capsule endoscopy 110 includes a capsule, an image sensor, a light emitting diode and an Ag₂O cell. In the prior art capsule endoscopy system 100, the capsule endoscopy 110 has a transmitter 112, and the data recorder 120 has a receiver 122 and a memory 124.

After swallowed by a patient, the capsule endoscopy 110 catches the image of the patient's digestive tract, and transmits the image to the receiver 122 of the data recorder 120 via the transmitter 112. Then the data is processed and stored in the memory 124.

Referring to FIGS. 1 and 2, after the data transmission and storage thereof, the data recorder 120 is wirelined to the image processor 130. The image processor 130 accesses the image data from the memory 124 for displaying the images for medical treatments.

From FIGS. 1 and 2, the prior art method includes two stages. In the first stage, the capsule endoscopy catches, transmits and stores the image of the digestive tract. In the second stage, the image is accessed and displayed. Because it takes about 8 hrs for the capsule endoscopy going through the digestive tract, the image thereof cannot be caught immediately. In other words, the prior are capsule endoscopy system cannot output image data to the image processor 130 during the first stage until it is finished. After the first stage is finished, the image data then can be displayed on the display 140.

### SUMMARY OF THE INVENTION

Therefore, an object of the present invention is to provide a capsule endoscopy system, which transmits the image of the digestive tract to the image processor immediately and display the image on the display for medical treatments.

The other object of the present invention is to provide a capsule endoscopy system, which transmits the image of the digestive tract to the image processor immediately via the transceivers triggered by the data recorder or the image processor thereof.

To achieve the objects above, the present invention discloses a capsule endoscopy system, adapted to transform an image of a digestive tract into an image data and to transmit the data, which comprises: a capsule endoscopy, a data recoder and an image processor. The capsule endoscopy has a first transceiver, wherein the capsule endoscopy is adapted to catch the image of the digestive tract and to transform the image into the image data. The data recorder has a second transceiver, a third transceiver and a memory, the second and the third transceivers coupled to the memory. Notably, the first transceiver of the capsule endoscopy transmits the image data to the second transceiver of the data recorder, which is stored in the memory and transmitted to the image processor by the third transceiver. In addition, the image processor above further comprises a fourth transceiver, adapted to receive the image data from the third transceiver.

To achieve the objects above, the present invention discloses a capsule endoscopy system, adapted to transform an image of a digestive tract into an image data and to transmit the data, which comprises: a capsule endoscopy, a data recoder, a fourth transceiver and an image processor coupled to the fourth transceiver. The capsule endoscopy has a first transceiver, wherein the capsule endoscopy is adapted to catch the image of the digestive tract and to transform the image into the image data. The data recorder has a second transceiver, a third transceiver and a memory, the second and the third transceivers coupled to the memory. Notably, the first transceiver of the capsule endoscopy transmits the image data to the second transceiver of the data recorder, which is stored in the memory and transmitted to the fourth transceiver and the image processor by the third transceiver.

According to the preferred capsule endoscopy system of the present invention, the transmission between the first and the second transceivers is continuous.

According to the preferred capsule endoscopy system of the present invention, the system further comprises a trigger, disposed in the data recorder or the image processor.

According to the preferred capsule endoscopy system of the present invention, the transmission between the third and the fourth transceivers is triggered by the trigger.

According to the preferred capsule endoscopy system of the present invention, the system further comprises a display coupled to the image processor for displaying the image of the digest tract.

In the capsule endoscopy system of the present invention, the image of the digestive tract is transmitted from the third transceiver to the fourth transceiver. After the processing of the processor, the image of the digestive tract can be displayed for medical treatment.

In order to make the aforementioned and other objects, features and advantages of the present invention understandable, a preferred embodiment accompanied with figures is described in detail below.

### BRIEF DESCRIPTION OF THE DRAWINGS

FIGS. 1 and 2 are transmission configuration of the prior art capsule endosocpy system.

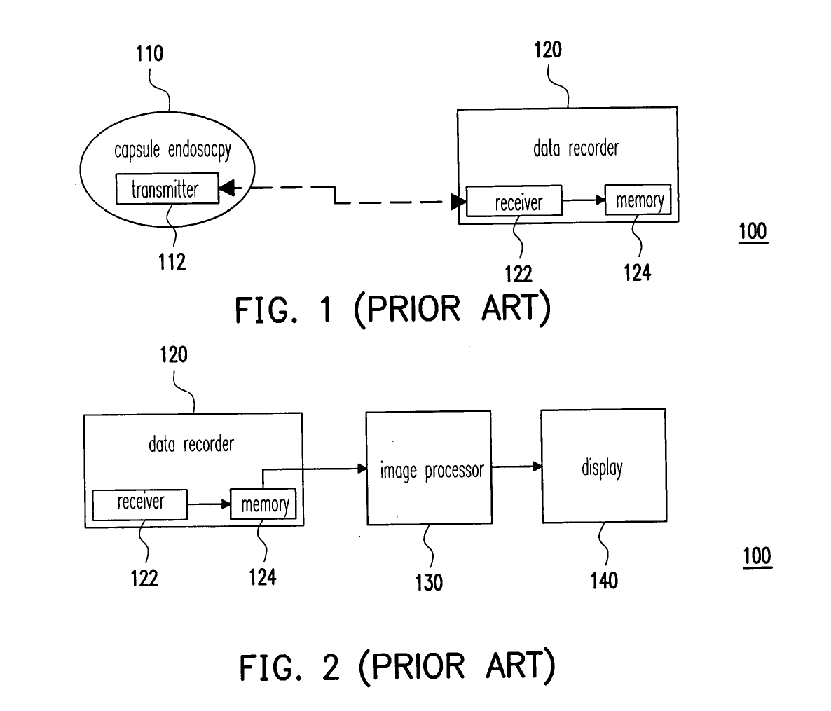

FIG. 3 is a schematic configuration showing an image data transmission of a preferred capsule endoscopy system of the present invention.

FIG. 4 is a schematic configuration showing an image data transmission of another preferred capsule endoscopy system of the present invention.

### DESCRIPTION OF SOME EMBODIMENTS

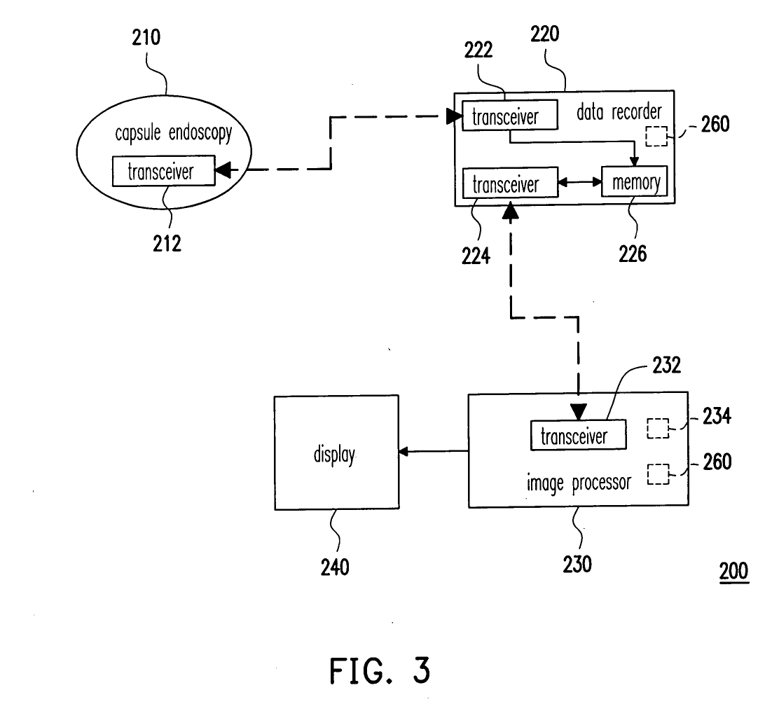

FIG. 3 is a schematic configuration showing an image data transmission of a preferred capsule endoscopy system of the present invention. Referring to FIG. 3, the capsule endoscopy system 200 is adapted to transform an image of a digestive tract into an image data and to transmit the data. Following are the descriptions of the capsule endoscopy system 200.

Referring to FIG. 3, the capsule endoscopy system 200 comprises: a capsule endoscopy 210, a data recoder 220 and an image processor 230. The capsule endoscopy 210 has a first transceiver 212, wherein the capsule endoscopy 210 is adapted to catch the image of the digestive tract and to transform the image into the image data. Additionally, the capsule endoscopy system 200 of the present invention further comprises a display 240 coupled to the image processor 230 for displaying the image of the digestive tract caught by the capsule endoscopy 210. The capsule endoscopy 210 can be, for example, a MIA capsule endoscopy (Given, Isreal), which comprises a light emitting diode, an image sensor, an Ag₂O cell, and a transceiver in a space 30 mm*11 mm. The capsule endoscopy 210, for example, catches two images for each second. Of course, the capsule endoscopy can be any other capsule endoscopies.

The data recorder 220 has a second transceiver 222, a third transceiver 224 and a memory 226, the second and the third transceivers 222 and 224, respectively, coupled to the memory 226. Notably, the first transceiver 212 of the capsule endoscopy 210 transmits the image data to the second transceiver 222 of the data recorder 220. In the embodiment, the transmission between the first and the second transceivers 212 and 222, respectively, is continuous. The image data received by the second transceiver 222 is stored in the memory 226 and transmitted to the image processor 230 by the third transceiver 224.

In the embodiment, the image processor 230 comprises, for example, a fourth transceiver 240, adapted to receive the image data from the third transceiver 224. Of course, the image processor 230 may, for example, comprise a memory 234 for storing the image data from the fourth transceiver 232. Notably, in order to reduce the transmission time, it is preferred that a high speed transmission is applied between the third and the fourth transceivers 224 and 232, respectively.

Referring to FIG. 3, the capsule endoscopy system 200 of the embodiment further comprises a trigger 260, adapted to transmit a signal or an order for triggering the transmission between the third and the fourth transceivers 224 and 232, respectively. In other words, the transmission between the third and the fourth transceivers 224 and 232, respectively, is triggered by the trigger 260. In addition, the trigger 260 is disposed, for example, in the data recorder 220 or the image processor 230. Of course, the trigger 260 can also disposed in the other position of the capsule endoscopy system 200.

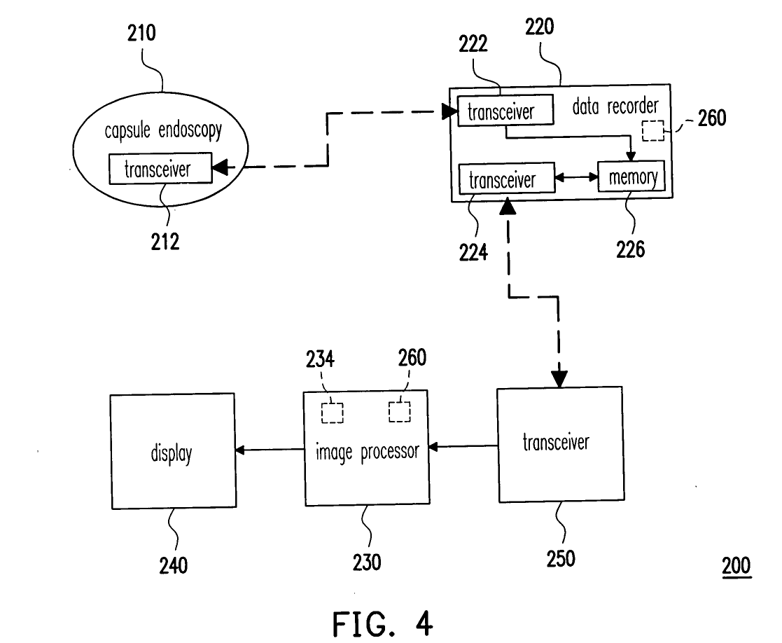

FIG. 4 is a schematic configuration showing an image data transmission of another preferred capsule endoscopy system of the present invention. Compared with FIGS. 3 and 4, the second embodiment is similar to the first embodiment. The difference is that the fourth transceiver 250 is out of the image processor 230 and coupled thereto.

In the embodiment, the fourth transceiver 250 serves receiving the image data from the third transceiver 224, and the image data received therefrom are stored in the memory 234 of the image processor 230.

Accordingly, the capsule endoscopy system of the present invention comprises following advantages:

1. The capsule endoscopy system of the present invention displays the image of the digestive tract immediately for medical treatments.

2. In the capsule endoscopy system of the present invention, the image of the digestive tract is transmitted from the third transceiver to the fourth transceiver. After the processing of the processor, the image of the digestive tract can be displayed for medical treatment.

Although the present invention has been described in terms of exemplary embodiments, it is not limited thereto. Rather, the appended claims should be constructed broadly to include other variants and embodiments of the invention which may be made by those skilled in the field of this art without departing from the scope and range of equivalents of the invention.

1. A capsule endoscopy system, adapted to transform an image of a digestive tract into an image data and to transmit the data, comprising:
a capsule endoscopy, having a first transceiver, wherein the capsule endoscopy is adapted to catch the image of the digestive tract and to transform the image into the image data;
a data recorder, having a second transceiver, a third transceiver and a memory, the second and the third transceivers coupled to the memory; and
an image processor, wherein the first transceiver of the capsule endoscopy transmits the image data to the second transceiver of the data recorder, which is stored in the memory and transmitted to the image processor by the third transceiver.

2. The capsule endoscopy system of claim 1, wherein the transmission between the first and the second transceivers is continuous.

3. The capsule endoscopy system of claim 1, further comprising a trigger, disposed in the data recorder or the image processor.

4. The capsule endoscopy system of claim 3, wherein the image processor has a fourth transceiver, adapted to receive the image data transmitted from the third transceiver.

5. The capsule endoscopy system of claim 4, wherein the transmission between the third and the fourth transceivers is triggered by the trigger.

6. The capsule endoscopy system of claim 1, further comprising a display coupled to the image processor for displaying the image of the digest tract.

7. A capsule endoscopy system, adapted to transform an image of a digestive tract into an image data and to transmit the data, comprising:
a capsule endoscopy, having a first transceiver, wherein the capsule endoscopy is adapted to catch the image of the digestive tract and to transform the image into the image data;
a data recorder, having a second transceiver, a third transceiver and a memory, the second and the third transceivers coupled to the memory;
a fourth transceiver; and
an image processor, wherein the first transceiver of the capsule endoscopy transmits the image data to the second transceiver of the data recorder, which is stored in the memory and transmitted to the fourth transceiver and the image processor by the third transceiver.

8. The capsule endoscopy system of claim 7, wherein the transmission between the first and the second transceivers is continuous.

9. The capsule endoscopy system of claim 7, further comprising a trigger, disposed in the data recorder or the image processor.

10. The capsule endoscopy system of claim 9, wherein the transmission between the third and the fourth transceivers is triggered by the trigger.

11. The capsule endoscopy system of claim 7, further comprising a display coupled to the image processor for displaying the image of the digest tract.
